(19) United States
(12) Patent Application Publication
(10) Pub. No.: US 2020/0038627 A1
(43) Pub. Date: Feb. 6, 2020

(54) Catheter desobstruction system and method

(71) Applicant: David Elsasser, Erlangen (DE)

(72) Inventor: David Elsasser, Erlangen (DE)

(21) Appl. No.: 16/524,930

(22) Filed: Jul. 29, 2019

(30) Foreign Application Priority Data
Jul. 31, 2018 (EP) EP18186489.3

(51) Int. Cl. A61M 25/00

(52) U.S. Cl. CPC A61M 25/0017; A61B 17/320758; A61M 2209/10; A61M 2025/0019

(57) ABSTRACT

A catheter desobstruction system for desobstructing a catheter, with a catheter desobstruction apparatus and a catheter desobstruction drive, wherein:
the proximal end of the flexible catheter cleaning cable is introduced into a lumen of the catheter;
the proximal end of the flexible catheter cleaning cable in moved into the proximal direction by the actor of the catheter desobstruction drive;
if a force detected by the force sensor of the catheter desobstruction drive exceeds a predetermined threshold, the flexible catheter cleaning cable is moved in the distal direction for a first predetermined distance by the actor;
when the flexible catheter cleaning cable has been moved the first predetermined distance in the distal direction, the proximal end of the flexible catheter cleaning cable is rotated by the motor of the catheter desobstruction drive and the flexible catheter cleaning cable is moved in the proximal direction by the actor.

The present invention relates to a catheter desobstruction apparatus, a catheter desobstruction drive and a use therefore, particularly for desobstructing urine catheters.

### FIELD OF THE INVENTION

Catheters for draining the bladder of a human are known since ancient times. Catheters are used, if a patient cannot drain his bladder. Catheters can be inserted transurethral or suprapubic.

A frequent problem in use of catheters is that the lumen of the catheter may get obstructed, such as by salt. Generally, such obstructions occur in the proximal portion of the catheter close to the inlet opening of the catheter.

If a permanent catheter gets obstructed, the patient will feel strong pain requiring acute intervention. In the prior art the catheter may be flushed back, e.g. by a syringe. In many cases the obstruction cannot be eliminated by flushing. Consequently, the catheter has to be changed under emergency conditions by a physician.

If the catheter is not changed as early as possible under emergency conditions, additional complications may develop by the patient, such as sepsis, kidney failure, inflammation of the urethra or the like.

Currently no catheter desobstruction apparatus for the desobstructing of an inserted catheter is commercially available.

### PRIOR ART

WO 87/02255 discloses an ultrasonic self-cleaning catheter system.

U.S. Pat. No. 5,030,213 discloses a catheter router assembly for cleaning salt blockages in a previously inserted catheter. A length of flexible, stainless steel cable is provided and a silver solder tip is applied at the proximal end of the cable. The silver solder tip is then machined to form a suitable cutting edge for cutting or drilling through the body salts which clog the catheter.

US 2005/0267421 A1 discloses a catheter cleaner comprising a head, a rod and a handle. The head defines a tip and cutting edges. The tip may have a curved configuration which may be operative to assist the insertion of the cleaner into an indwelling catheter.

U.S. Pat. No. 6,183,450 B1 discloses a catheter cleaning device for cleaning a catheter of obstructions. The catheter cleaning device comprises an elongate hollow housing, a guide member encircling the housing and including a bar extending across a diameter thereof and through first and second slots of the elongate hollow housing. A guide wire extends from the bar.

### SUMMARY OF THE INVENTION

It is an object of the present invention to provide a catheter desobstruction apparatus for desobstructing a catheter inserted into a patient.

The object of the present invention is solved by a catheter desobstruction apparatus according to claim 1, a desobstruction drive according to claim 9, a catheter desobstruction system according to claim 13 and a use of the catheter desobstruction system according to claim 14. The depending claims claim preferred embodiments.

The invention discloses a catheter desobstruction apparatus comprising a flexible catheter cleaning cable comprising at its proximal end a desintegration tool and at its distal end a cable coupling. The flexibel catheter cleaning cable is adapted to be introduced into a lumen of a catheter. The catheter desobstruction apparatus further comprises a drain element having a first opening, a second opening and a third opening. The first opening is adapted to be connected to the catheter. The first and second opening are adapted that the catheter cleaning cable can pass through the first and second opening. The third opening is in fluid communication with the first opening.

The flexible catheter cleaning cable can be introduced into the lumen of the catheter for removing the obstruction in the lumen of the catheter. As soon as the obstruction has been removed, urine may flow from the bladder to the drain element. The drain element directs the urine to the third opening to which a tube, a pipe, a bag and/or a container may be connected.

The disintegration tool can be a blade, a drill, a crushing tool or the like.

At the first, the second and third opening a Luer cone may be arranged.

The present invention has the advantage that urine released from the bladder into the catheter flows in a determined way to a vessel without contaminating the bed of the patient, the operating table or the like.

The first opening may be arranged opposite to the second opening. In this embodiment the flexible catheter cleaning cable passes in a straight manner through the drain element.

The drain element may have a generally T-shaped configuration. The first opening and the second opening may be arranged opposite to each other. The third opening may be arranged between the first and second opening and/or perpendicular to the first and second opening.

The flexible catheter cleaning cable may pass through the first and second opening.

The drain element and the flexible catheter cleaning cable may be located in a sterile packing. The flexible catheter cleaning cable passes through the first and second opening, such that the catheter desobstruction apparatus is ready for use after opening the sterile packing.

The catheter desobstruction apparatus may further comprise a sealing arranged in use around the flexible catherter cleaning cable blocking flow of a liquid (e.g. urine) from the first opening to the second opening.

In one embodiment the drain element comprises a first conduit extending from the first opening to the second opening and a second conduit branching off from the second conduit, wherein the sealing is arranged between the second opening and the branch off of the conduit. The sealing does not have to extend from the second opening to the branch off of the second conduit. Thereby, release of urine by the second opening can be prevented. The urine will be released by the drain element only by the third opening.

The invention also discloses a catheter desobstruction drive having a drive coupling adapted to be connected with a cable coupling of a flexible catheter cleaning cable. The catheter desobstruction drive comprises a motor rotatory driving the drive coupling. The drive coupling may rotate about a rotation axis passing through the center of the drive coupling. The catheter desobstruction drive may further comprise an actor translationally moving the drive coupling. The drive coupling may be moved along the rotation axis of the drive coupling. The catheter desobstruction drive may further comprise a force sensor adapted to determine the force acting on the drive coupling in the translational direction. The catheter desobstruction drive further comprises a controller adapted to control the motor and actor.

The catheter desobstruction drive may comprise an opening through which the flexible catheter cleaning cable can pass and a sealing adjacent to the opening through which the flexible catheter cleaning cable can pass and preventing that a liquid can enter the drive. The sealing may comprise bellows through which the catheter cleaning cable passes.

In one embodiment, the controller is adapted to instruct the motor to rotate the drive coupling and the actor to move the drive coupling in the proximal direction. Thereby, the obstruction is removed by the rotating desintegration tool at the distal end of the flexible catheter cleaning cable around its longitudinal axis.

In another embodiment, the controller is adapted to instruct the actor to move the drive coupling into the proximal direction and to instruct the motor not to rotate the drive coupling. If a force detected by the force sensor exceeds a predetermined threshold, the controller instructs the actor to move the drive coupling in the distal direction for a first predetermined distance. If the force detected by the force sensor exceeds a predetermined threshold, the desintegration tool of the flexible catheter cleaning cable might have contacted the obstruction. Thereafter, the desintegration tool is removed from the obstruction by moving the flexible cleaning cable from the obstruction, when the catheter desobstruction drive is connected to the above described catheter desobstruction apparatus. When the actor has moved the drive coupling the first predetermined distance in the distal direction, the controller instructs the motor to rotate the drive coupling and the actor to move the drive coupling in the proximal direction. Thereby, the obstruction is removed by the rotating desintegration tool at the distal end of the flexible catheter cleaning cable around its longitudinal axis. In this embodiment the disintegration tool only rotates in the portion close to the obstruction. Thereby, damaging of the catheter may be avoided.

The controller may be further adapted to instruct the actor to move the drive coupling a predetermined distance in the proximal direction and the motor to rotate the drive coupling. Thereby, the obstructions in the lumen of catheter are removed by the rotating desintegration tool at the proximal end of the flexible catheter cleaning cable. If the actor has moved the drive coupling the predetermined distance in the proximal direction, the controller instructs the actor to move the drive coupling a second predetermined distance in the distal direction and the motor to rotate the drive coupling. The flexible catheter cleaning cable is moved partially outward of the catheter and the desintegration tool at the distal end of the flexible catheter cleaning cable passes again the location which was obstructed. By the rotating desintegration tool residues of the obstruction are removed. Since the flow of urine is not blocked after removing the obstruction the urine is released from the bladder and flows through the catheter to a container connected to the third opening. Thereby, any residues of the obstruction in the lumen of the catheter are flushed.

The predetermined distance may be a set distance. The predetermined distance may be computed dynamically, e.g. based on a force exerted on the actor and/or motor. The force exerted on the actor and/or motor may fall under a predetermined threshold, after the obstruction is removed by the disintegration tool.

The actor may be an electric actor and the force sensor may be a current sensor measuring the current supplied to the electric actor.

The invention also discloses a catheter desobstruction system comprising the catheter desobstruction apparatus described above and the catheter desobstruction drive described above.

The present invention also discloses a use of the catheter obstruction system for desobstructing a catheter. The proximal end of the flexible catheter cleaning cable is introduced into a lumen of the catheter. This step may be performed by nursing staff. Thereafter, the drive coupling of the catheter desobstruction drive is coupled with cable coupling of the flexible catheter cleaning cable of the catheter desobstruction apparatus, e.g. by the nursing staff. Then, the catheter desobstructing method is activated by pressing a button on the desobstruction drive. The controller of the catheter desobstruction device controls the catheter desobstruction method.

In one embodiment, the proximal end of the flexible catheter cleaning cable is then rotated by the motor of the catheter desobstruction drive and the flexible catheter cleaning cable is moved in the proximal direction by the actor. Thereby, the obstruction in the lumen of the catheter is removed.

According to another embodiment, the proximal end of the flexible catheter cleaning cable is moved into the proximal direction by the actor of the catheter desobstruction drive without rotating the distal end. If a force detected by the force sensor of the catheter desobstruction drive exceeds a predetermined threshold, the flexible catheter cleaning cable is moved in the distal direction for a first predetermined distance by the actor. When the flexible catheter cleaning cable has been moved the first predetermined distance in the distal direction, the proximal end of the flexible catheter cleaning cable is rotated by the motor of the catheter desobstruction drive and the flexible catheter cleaning cable is moved in the proximal direction by the actor. Thereby, the obstruction in the lumen of the catheter is removed.

If the flexible catheter cleaning cable has been moved the predetermined distance in the proximal direction, the flexible catheter cleaning cable is moved a second predetermined distance in the distal direction by the actor and the flexible catheter cleaning cable is rotated by the motor. Thereby, residues of the removed obstruction are removed. Further, the residues of the obstruction are flushed by the urine released from the bladder to the third opening of the drain element and a device connected thereto. If the flexible catheter cleaning cable has been moved the second predetermined distance in the distal direction, moving of the flexible catheter cleaning cable is stopped and rotating of the proximal end of the flexible catheter cleaning cable is stopped.

The predetermined distance may be a set distance. The predetermined distance may be computed dynamically, e.g. based on a force exerted on the actor and/or motor. The force exerted on the actor and/or motor may fall under a predetermined threshold, after the obstruction is removed by the disintegration tool.

Thereafter, a service staff can remove the flexible catheter cleaning cable from the lumen of the catheter.

The present invention provides the advantage that an obstruction in an inserted catheter can be removed by nursing staff and no supervision by a physician is necessary. Since the inventive apparatus may be operated by nursing staff, the obstruction in the catheter can be removed within a short period after the obstruction occurred. Thereby, comfort of the patient is increased and additional complications described above are avoided.

### BRIEF DESCRIPTION OF THE DRAWINGS

The invention is now explained with reference to the drawings showing non-limiting embodiments of the invention, wherein

FIG. 1 is a schematic view of a catheter desobstructing system;

FIG. 2 show a catheter desobstructing apparatus in a sterile packing; and

FIG. 3 shows a flowchart of the use of the present invention.

### DETAILED DESCRIPTION OF THE DRAWINGS

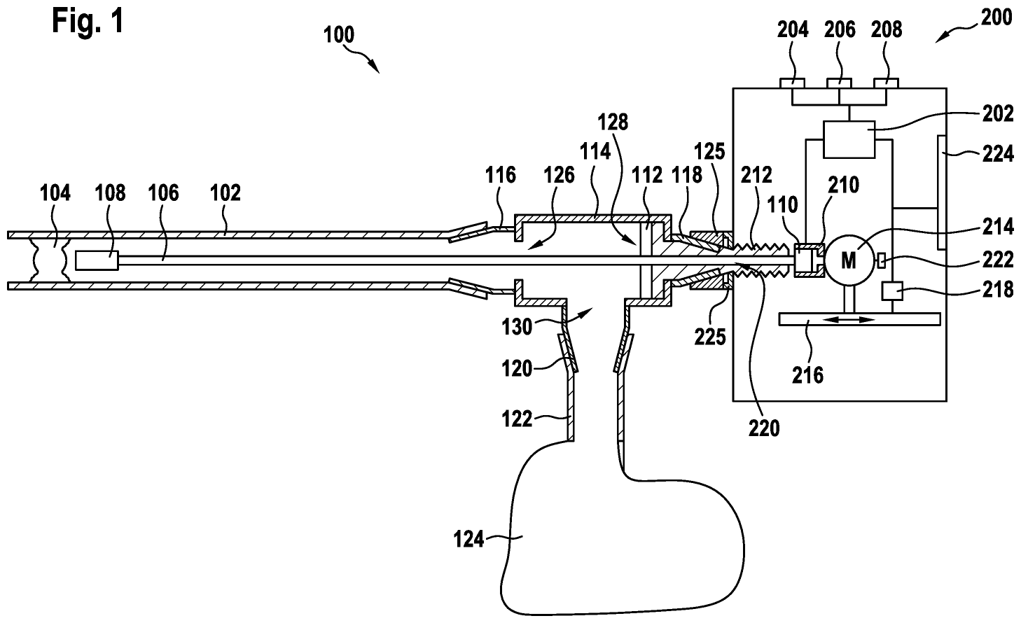

FIG. 1 depicts a schematic view of the catheter desobstruction system comprising the catheter desobstruction apparatus 100 and the catheter desobstruction drive 200. The desobstruction apparatus 100 comprises a cable 106, i.e. a flexible catheter cleaning cable, wherein at the proximal end of the cable 106 a blade 108 is arranged. The blade 108 is an example of a desintegration tool and alternatively to the blade a crushing tool, a drill or the like may be used.

The proximal end of the catheter 102 is positioned releasably in the bladder of the patient such that the patient can release urine. The catheter 102 has been inserted by a physician, if a patient cannot release urine.

In use, if a catheter is obstructed, the cable 106 and the blade 108 are inserted into the lumen of a catheter 102 by a user, such as nursing staff. The catheter 102 is obstructed by salt 104 such that urine cannot flow from the bladder of a patient through the catheter 102.

The distal end portion of the catheter 102 is pushed by the user on a first cone 116 adjacent to a first opening 126 of a drain element 114. The drain element 114 comprises the first cone 116 adjacent to the first opening 126, a second cone 118 adjacent to a second opening 128 and a third cone 120 adjacent to a third opening 113. A tube 122 connected to a bag 124 is connected to the third cone 120. Urine can flow from the catheter 102 through the first cone 116, the first opening 126, the third opening 113 and the third cone 120 of the drain element 114 to the tube 122 and the bag 124.

In one embodiment the first cone 116, the second cone 118 and/or the third cone 120 may be a Luer cone.

The cable 106 extends through the first cone 116, the first opening 126, the second opening 128 and the second cone 118 of the drain element 114. The first cone 116 and the second cone 118 are arranged opposite to each other. The third cone 120 is arranged essentially perpendicular to the first cone 116 and the second cone 118. The drain element 114 is generally T-shaped. In a passage between the third cone 120 and the second cone a sealing 112 is arranged around the cable 106 for avoiding that a fluid can flow to the third cone 118.

To the second cone 118 a connector 125 for connecting the drain element 114 to a drive 200 explained below is arranged.

The drive 200 is adapted to rotate the cable 106 around its longitudinal axis and to move the cable along its longitudinal axis. The drive 200 comprises a motor 214 for rotating the cable around its longitudinal axis and a carriage 216 for displacing the cable 106 along its longitudinal axis. The motor 214 is connected to a controller 202 by the motor controller 222, and the carriage 216 is connected via a carriage controller 218 to the controller 202.

The controller is connected to a power-on switch 204, a start switch 206 and a release switch 208. If a user presses on the power-on switch 204, the drive is supplied with power and activated. As soon as the user presses the start button 206 an automatic method for desobstructing the catheter 102 is started as described below in detail.

The drive 200 further comprises a complimentary connector 225 for receiving the connector 125 of the drain element 114. The connector 125 of the drain element 114 and the complimentary connector 225 of the drive define the positional relationship between the drive 200, the drain element 114 and the cable 106.

The cable 106 comprises at its distal end a cable coupling 110. In one embodiment the cable coupling 110 may have a square cross section. The cable coupling 110 of the cable 106 is received by a drive coupling 210. The drive coupling 210 may be a clamp, a clamp jug, a clamping nut or the like that may be operated by the controller 202.

The drive further comprises bellows 212 or a suitable seal arranged at the opening 220 adjacent to the complimentary connector 225 for avoiding that a liquid can enter the drive, i.e. if the seal 112 fails. The cable 106 may pass in its longitudinal direction (expansion direction of the bellows) through the bellows. The bellows can be expanded or contracted, if the carriage 216 moves the motor 214 and the drive coupling 210 and the cable coupling 110 as well as the cable 106 connected thereto.

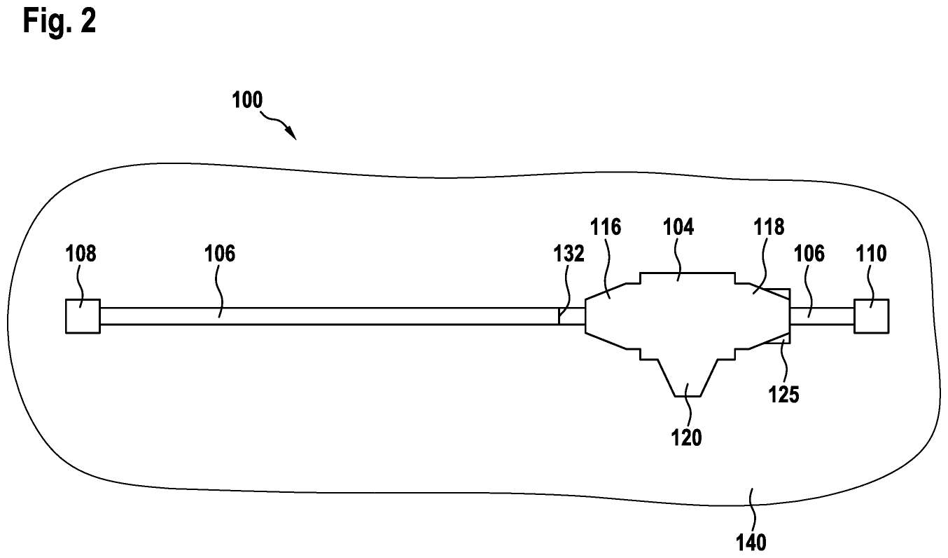

FIG. 2 shows the catheter desobstruction apparatus 100, as it is delivered to a user, such as a nursing staff. The desobstruction apparatus 100 is accommodated in a sterile packing 140. The cable 106 runs from the first cone 116 to the second cone 118. The blade 108 is arranged at the proximal end of the cable 106 and the cable coupling 110 is arranged at the distal end of the cable 106. The connector 125 is arranged on the second cone 118.

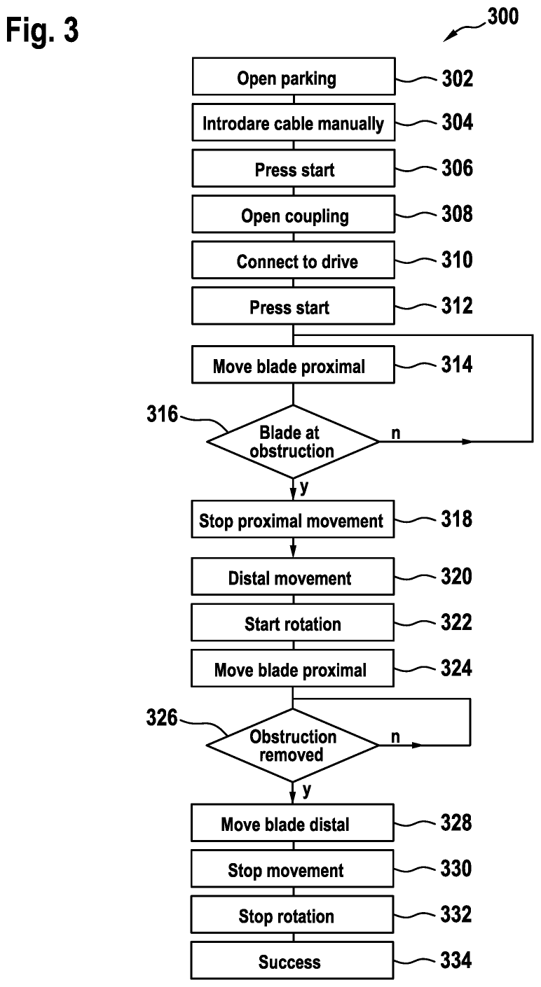

With reference to FIG. 3 a method 300 for using the catheter desobstruction system 100, 200 is described with respect to a urine catheter 102. In a first step 302 a user, such as nursing staff opens the sterile packing 140. Then, the user introduces the blade 108 and the cable 106 up to a predetermined position indicated by a marking 132 into the catheter 102 in step 304. The obstructions 104 in a urine catheter are generally formed by salt and are generally located close to the proximal opening of the catheter 102.

Then, in step 306 the user presses the start button 206 of the drive after having powered on the drive by pressing the power on button 204. The controller 202 instructs the drive coupling 210 to open in step 308. Thereafter, in step 310 the user connects the desobstruction apparatus 100 with the drive 200. Thereby, the cable connector 110 is positioned in the drive connector 210 and the connector 125 of the drain element 114 is releasably connected with the complimentary connector 225 of the drive 200.

In the following step 312 the user presses the start button again. The controller 202 instructs the drive coupling 210 to clamp the cable coupling 110 fixedly.

In step 314 the automatic desobstruction procedure commences. Particularly, in step 314 the controller 202 instructs the carriage 216 to move the blade 108 in the proximal direction. The carriage controller 218 monitors the current supplied to the carriage 216. If the current detected by the carriage controller 218 is below a predetermined threshold in step 316 the controller instructs the carriage 216 to move the blade 108 in the proximal direction. As soon as the carriage controller 218 detects in step 316 that the current supplied to the carriage 216 exceeds a predetermined threshold, the controller 202 stops the movement of the carriage 216 in step 318. The current supplied to the carriage 216 is increased, because the blade 108 touched the obstruction 104.

Then, the controller 202 proceeds with step 320 and instructs the carriage 216 to move the blade 108 a first predetermined distance in the distal direction.

As soon as the carriage 216 has moved the blade 108 the first predetermined distance in the distal direction, the controller 202 instructs the carriage 216 to stop movement in the distal direction. Thereafter, the controller 202 instructs in step 322 the motor 214 to rotate the blade 108 around the longitudinal axis of the cable 106. The torque of the motor 214 is transferred by the drive coupling 210 to the cable coupling 110. In order to minimize friction of the cable 106 in the catheter 102, the cable 106 may be coated with Teflon or the like. In order to minimize friction, the cable may comprise cylindrical guides arranged along the cable 106. In another embodiment the cable 106 may be coated with silicone or plastics. Further, the controller 202 instructs the carriage 216 to move the blade 108 in the proximal direction in step 324.

As soon as the blade 108 touches the obstruction 104, the current supplied to the motors 214 will increase as detected by motor controller 222, due to the friction caused by the obstruction 104. Further, the current supplied to the carriage 216 increases as detected by the carriage controller 218 due to the friction caused by the obstruction 104. At least one of the current supplied to the motor 214 and the current supplied to the carriage 216 is monitored in step 326. As long as the current supplied to the motor 214 and/or the current supplied to the carriage 216 remain above a predetermined threshold, the obstruction 104 is removed by the blade and the blade 108 is continued to be rotated and moved in the proximal direction.

As soon as the controller 202 detects that the current supplied to the motor 214 and/or carriage 216 falls under the predetermined threshold, the obstruction 104 has been removed by the blade. The controller proceeds to step 328. Further, as soon as the obstruction 104 is removed, urine flows through the catheter and the first opening 126 and the third opening 130 of the drain element 114 into the bag 124.

In step 328 the controller 202 instructs the carriage 216 to move the blade 108 in the distal direction and the motor 214 to rotate the blade. Thereby, debris of the obstruction 104 can be removed from the catheter 102.

As soon as the carriage 216 has moved the blade 108 a second predetermined distance in the distal direction, the controller 202 instructs in step 330 the carriage 216 to stop the movement in the distal direction. Thereafter, in step 332 the controller 202 instructs the motor 214 to stop rotating.

The desobstruction method has been finalized successfully and a message indicating success of the desobstruction method is displayed on the display 224 of the drive 200. If a user presses the release switch 208, the controller instructs the drive coupling 210 to release the cable coupling 110 and the cable is disconnected from the cleaning device.

The user removes the cable 106 and the drain element 104 from the catheter 102 and removes the drive 200 from the drain element 114, after the controller has instructed the drive coupling 210 to release the cable coupling 110 after successfully finalizing the desobstruction method.

The drive 200 may be reused. The desobstruction apparatus 100 can be discarded or cleaned and sterilized for further usage.

As soon as the drain element 114 is removed from the catheter 102 the urine bag of the patient can be connected to the catheter.

The present invention provides the advantage that an acute obstruction of a urine catheter 102 can be desobstructed by nursing staff within a short time span. Thereby, pain of the patient is avoided, and additional complications such as sepsis, kidney failure, inflammation of the urethra or the like of the patient may be avoided. Further, costs can be reduced significantly since medical intervention by a physician or transportation of the patient to hospital can be avoided.

1. A catheter desobstruction apparatus, comprising
a flexible catheter cleaning cable comprising at its proximal end a desintegration tool and at its distal end a cable coupling, wherein the flexible catheter is adapted to be introduced into a lumen of a catheter;
a drain element having a first opening, a second opening and a third opening;
wherein
the first opening is adapted to be connected to the catheter;
the first and second opening are adapted that the cleaning cable can pass through the first and second opening;
the third opening is in fluid communication with the first opening.

2. The catheter desobstruction apparatus according to claim 1, wherein the third opening is adapted to at least one of the following:
to be connected to a tube;
to be connected to a pipe;
to be connected to a bag;
to be connected to a container.

3. The catheter desobstruction apparatus according to claim 1, wherein the first opening is opposite to the second opening.

4. The catheter desobstruction apparatus according to claim 1, wherein the drain element has a generally T-shaped configuration.

5. The catheter desobstruction apparatus according to claim 1, wherein the flexible catheter cleaning cable passes through the first opening and second opening.

6. The catheter desobstruction apparatus according to claim 1, wherein the drain element and the flexible catheter cleaning cable are located in a sterile packaging.

7. The catheter desobstruction apparatus according to claim 1, further comprising a sealing arranged in use around the flexible catheter cleaning cable blocking flow of a liquid from the first opening to the second opening.

8. The catheter desobstruction apparatus according to claim 7, wherein the drain element comprises a first conduit extending from the first opening to the second opening and a second conduit branching off from the second conduit, wherein the sealing is arranged between the second opening and the branch off of the second conduit.

9. A catheter desobstruction drive, having
drive coupling adapted to be connected with a cable coupling of a flexible catheter cleaning cable;
a motor rotatory driving the drive coupling;
an actor translationally moving the drive coupling;
a force sensor adapted to determine the force acting on the drive coupling in the translational direction; and
a controller adapted to control the motor and actor;
wherein the controller is adapted to the following:
instructing the motor to rotate the drive coupling and the actor to move the drive coupling in the proximal direction.

10. The catheter desobstruction drive of claim 9, further comprising:
an opening through which the flexible catheter cleaning cable can pass; and
a sealing adjacent to the opening through which the flexible catheter cleaning cable can pass and preventing that a liquid can enter the drive.

11. The catheter desobstruction drive of claim 9, wherein the controller is further adapted to the following:
instructing the actor to move the drive coupling a predetermined distance in the proximal direction and the motor to rotate the drive coupling;
if the actor has moved the drive coupling the predetermined distance in the proximal direction, instructing the actor to move the drive coupling a predetermined distance in the distal direction and the motor to rotate the drive coupling; and
if the actor has moved the drive coupling the second predetermined distance in the distal direction, instructing the actor to stop moving the drive coupling and the motor to stop rotating the drive coupling.

12. The catheter desobstruction drive of claim 11, wherein the actor is an electric actor and the force sensor is a current sensor measuring the current supplied to the electric actor.

13. A catheter desobstruction system comprising the catheter desobstruction apparatus according to claim 1 and the catheter desobstructing drive according to claim 9.

14. Use of the catheter desobstruction system according to claim 13 for desobstructing a catheter, comprising the following steps:
introducing the proximal end of the flexible catheter cleaning cable into a lumen of the catheter; and
rotating the proximal end of the flexible catheter cleaning cable by the motor of the catheter desobstruction drive and moving the flexible catheter cleaning cable in the proximal direction by the actor.

15. Use according to claim 13, further comprising the following steps:
moving the flexible catheter cleaning cable a predetermined distance in the proximal direction by the actor and rotating the proximal end of the flexible catheter cleaning cable by the motor;
if the flexible catheter cleaning cable has been moved the predetermined distance in the proximal direction, moving the flexible catheter cleaning cable a predetermined distance in the distal direction by the actor and rotating the flexible catheter cleaning cable by the motor; and
if the flexible catheter cleaning cable has been moved the second predetermined distance in the distal direction, stop moving the flexible catheter cleaning cable and stop rotating the proximal end of the flexible catheter cleaning cable.
